(19) United States
(12) Patent Application Publication
(10) Pub. No.: US 2025/0236655 A1
(43) Pub. Date: Jul. 24, 2025

(54) PROCESS FOR THE PREPARATION OF LIRAGLUTIDE

(71) Applicants: BHARTI PRAFULLA DESHMUKH, RAICHUR (IN); VIJAY PETHA, RAICHUR (IN)

(72) Inventors: BHARTI PRAFULLA DESHMUKH, RAICHUR (IN); VIJAY PETHA, RAICHUR (IN)

(73) Assignee: SHILPA PHARMA LIFESCIENCES LIMITED, Raichur (IN)

(21) Appl. No.: 18/420,778

(22) Filed: Jan. 24, 2024

(51) Int. Cl. C07K 14/605; C07K 1/04; C07K 1/06; C07K 1/20

(52) U.S. Cl. CPC C07K 14/605; C07K 1/042; C07K 1/045; C07K 1/064; C07K 1/20

(57) ABSTRACT

The present invention relates to an improved process for the preparation of Liraglutide.

The present invention further related an improved process for the preparation of substantially pure material having a purity of greater than or equal to 99.5% by HPLC, wherein the process involves hybrid technology of both manual and microwave to produce Liraglutide.

### FIELD OF THE INVENTION

The present invention further related an improved process for the preparation of substantially pure material having a purity of greater than or equal to 99.5% by HPLC, wherein the process involves hybrid technology of both manual and microwave to produce Liraglutide.

### BACKGROUND OF THE INVENTION

Liraglutide is marketed under the brand name VICTOZA® in the U.S., India, Canada, Europe and Japan. The peptide precursor of liraglutide, produced by a process that includes expression of recombinant DNA in Saccharomyces cerevisiae, has been engineered to be 97% homologous to native human GLP-1 by substituting arginine for lysine at position 34. Liraglutide is made by attaching a C-16 fatty acid (palmitic acid) with a glutamic acid spacer on the remaining lysine residue at position 26 of the peptide precursor. The molecular formula of liraglutide is C₁₇₂H₂₆₅N₄₃O₅₁ and the molecular weight is 3751.2 Daltons. The structural formula (FIG. 1) is:

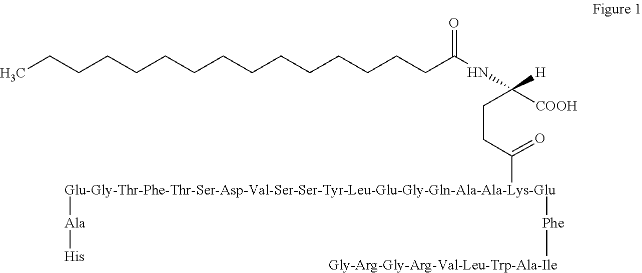

As per the scientific discussion “The liraglutide drug substance manufacturing process has adequately been described and a flow chart has been provided. Briefly, it consists of the following main steps: fermentation of yeast cells, recovery and purification of liraglutide precursor, acylation of the precursor, and further purification of liraglutide to drug substance.”

Liraglutide is first disclosed in U.S. Pat. Nos. 6,268,343B1 and 6,458,924B2, in which Liraglutide preparation by biological route, mainly through genetic engineering and other biological methods of preparation. Liraglutide preparation by a biological route involves technical difficulties and limitations in the attachment of the Palmitoyl-Glu-spacer.

The process generates biological impurities including unwanted proteins, cell debris, host DNA, and genetic material which required immense purification processes, therefore production costs increase. The disadvantage of the process described in US '343 is that the N-terminal of GLP-1(7-37)-OH is not protected, which leads to the generation of impurities. Additional purification steps are required to remove these impurities, and it makes Liraglutide high cost and not suitable for large-scale production.

US '343 and US '924 disclosed a process for the purification of Liraglutide, wherein the purification is performed using reverse-phase HPLC for intermediate GLP-1(7-37)-OH, followed by reaction with Nα-alkanoyl-Glu(ONSu)-OtBu in the liquid phase. In such a process, the N-terminal of GLP-1(7-37)-OH is not protected and protective groups for the side chains are all removed, leading to the formation of a great number of impurities, difficulties in purification, and complicated operation steps. The prior art process for the preparation of the liraglutide involves purification steps, a long synthesis cycle, and a large amount of waste liquid which is not environmentally friendly and involves a high amount of solvent like acetonitrile, which is cumbersome in large-scale production.

U.S. Pat. No. 7,572,884 disclosed a process for preparing Liraglutide using recombinant technology followed by acylation and removal of N-terminal extension.

U.S. Pat. No. 9,260,474 disclosed a process for the preparation of solid phase synthesis of Liraglutide comprises
a) the presence of the activator system, solid phase carrier, and by resin Fmoc protection N end obtained by coupling of glycine (Fmoc-Gly-OH) Fmoc-Gly-resin;
b) by solid phase synthesis, prepared in accordance with the sequentially advantage Liraglutide principal chain N end of the coupling with Fmoc protected amino acid side chain protection and, wherein the lysine using Fmoc-Lys (Alloc)-OH;
c) Alloc getting rid of the lysine side chain protecting group;
d) by solid phase synthesis, the lysine side chain coupling Palmitoyl-Glu-OtBu;
e) cracking, get rid of protecting group and resin to obtain crude Liraglutide;
f) purification, freeze-dried, to obtain Liraglutide.

Even though the above-mentioned prior art discloses diverse processes for the preparation of Liraglutide, they are often not amenable on a commercial scale because of expensive amino acid derivatives such as pseudo prolines used in those processes.

International publications WO 2019/170918 and WO 2019/170895 disclosed a process for the preparation of liraglutide comprising enzymatically coupling of a peptide C-terminal ester or thioester comprising a first peptide fragment with a peptide nudeophile having an N-terminally unprotected amine comprising a second peptide fragment, wherein enzyme coupling is catalyzed by ligase.

As the process described in this patent application involves the use of enzyme ligase, which additionally requires purification of the enzyme step and is difficult to handle the content of enzyme in the final liraglutide.

WO 2016/046753 discloses methods for synthesizing GLP-1 peptides, including Liraglutide and Semaglutide, which comprise a final coupling step in which at least two fragments are coupled at a terminal Gly residue, wherein at least one of the fragments is prepared by the coupling of at least two sub-fragments. By way of example, WO 2016/046753 discloses coupling fragments (1-4) and fragments (5-31) in solid state or in solution. Fragments (5-31) can be prepared by coupling fragments (5-16) with fragments (17-31). Fragment (5-16) itself can be prepared by coupling fragment (5-12) with fragment (13-16). Coupling with a terminal Gly, for example, at Gly4 or Gly16, avoids racemization.

WO 2019/153827 disclosed a process for the preparation of a liraglutide intermediate polypeptide GLP-1(7-37), comprising constructing recombinant liraglutide engineered bacteria, expressing a liraglutide intermediate fusion protein in the form of an inclusion body by means of E. coli induction. The prepared liraglutide intermediate polypeptide has a purity reaching 87% or higher and a yield greater than 85%.

U.S. Pat. No. 10,344,069 disclosed a process for the preparation of Liraglutide, which comprises the synthesis of suitable fragments (protected) by solid phase peptide synthesis; followed by coupling of the suitable fragments on a solid support; concurrently cleaving the protected peptide from the solid support and de-protecting the peptide; followed by purification of Liraglutide (crude) on reverse phase HPLC and isolating pure Liraglutide.

Further, WO 2016/005960 and WO 2019/069274 disclosed a process for the preparation of liraglutide comprised of sequential development of fragments followed by coupling.

The main disadvantage of this fragment coupling, fragment condensation into each solid phase segment needed excess fragments to condensation, it resulted in a serious waste of peptide fragments hence it resulting into the high cost of synthesis. Solid phase segment condensation needed synthesis in multiple reactions and its condensation having limitation in resin substitution, and it generate large amount of waste. Also the solid phase fragment condensation method of synthesis generates impurities of fragments during the condensation due to unreacted fragments and it also leads to the formation of optically impure Liraglutide and it is difficult to purify to its homogeneity

In Patent WO2013/037266 describes solid phase synthesis of Liraglutide synthesis by using Alloc-protected Lysine in a linear sequence. After completion of the liraglutide peptide sequence attachment of the peptide linker is a must to complete the molecule. The patent described the use of tetrakis (triphenylphosphine) palladium to remove Alloc and then attachment of the Peptide linker. This process is not cost-effective due to the use of tetrakis (triphenylphosphine) palladium to deprotect lysine.

In Patent WO2014/199397 describes the use of Dde-protected Lysine substrate and it's selective deprotection by using hydrazine. As hydrazine can also remove Fmoc groups as well as Dde groups. The basic nature of hydrazine removes Fmoc protections as lead to form unexpected side impurities during synthesis if traces of hydrazine in synthesis.

In Patent WO2015/100876 describe resin solid phase carrier is 2-CTC resin and activated system selected from the DIEA, TMP or NMM for CTC resin and DIC, HOBt and DMAP for king resin, and the Fmoc-Gly resin is 0.10-0.35 mmol./g Substitution degree of Fmoc-Gly on both resins, the lower substitution of Fmoc Gly on resin which results in low yield. Further in the same patent disclosed the molar ratio as 1:3:3:3:3 and 1:5:5:5:5, it clear indicates that amino acids are is used in large excess which results into higher production cost.

Chinese patent CN 103145828 B describe the sequential coupling of solid-phase peptide synthesis of liraglutide, using 2-chlorotrityl resin as a solid-phase carrier, using DIC/HOBt as a condensing agent on specific microwave reaction technology. In this process the lysine is protected by the ivDde side chain, after completion of linkage of liraglutide peptide sequence, the ivDde group had been deprotected, followed by inclusion of Palmitoyl side chain.

This technology reduces the reaction time and increased the condensation efficiency. The more hindered 1-(4,4-dimethyl-2,6-dioxocyclohex-1-ylidene)isovaleryl (ivDde) group was introduced to overcome migration issue. However, this process involves tedious work up for the removal of IvDde, which is commercially not feasible and involves the formation of major front and back impurities.

CN 106397573 discloses solid-peptide synthesis of liraglutide with the use of specific microwave reaction technology. The solid-phase synthesis involves the use of 2-Chloro trityl resin as a starting resin carrier under specific microwave technology, corresponding amino acids in a liraglutide sequence are connected in sequence to obtain Fmoc-liraglutide-2-Chlorotrityl-Resin. In this process the lysine is protected by the Dde side chain, after completion of linkage of liraglutide peptide sequence, the Dde group had been deprotected, followed by inclusion of Fmoc-Glu (OtBu)-OH and a palmitic acid is connected to a glutamic acid to obtain liraglutide-2-Chloro trityl-Resin.

This technology reduces the reaction time and increased the condensation efficiency. The Dde group has been observed to undergo migration during deprotection of NF-Fmoc-protected lysine residues and N-terminal Dpr residues. In the synthesis of long sequences, partial loss of Dde protecting groups may give side impurity which will may hamper purification has been reported.

Hence, there remains a need to provide simple, cost effective, scalable and robust processes for the preparation of Liraglutide involving commercially viable amino acid derivatives and reagents.

In view of the above it is pertinent to note that there is a need to develop new process for the preparation of Liraglutide having further improved physical and/or chemical properties besides high purity levels. Hence it was thought worthwhile by the inventors of the present application to explore novel process for the preparation of Liraglutide, which may further improve the characteristics of drug Liraglutide and in developing the substantially pure Liraglutide.

Exploring new process for developing a stable and pure form of Liraglutide, which are amenable to scale up for pharmaceutically active useful compounds in the preparation of Liraglutide may thus provide an opportunity to improve the drug performance characteristics of products such as purity and solubility. Hence, inventors of the present application report a process for the preparation of a stable and substantially pure form of Liraglutide, which may be industrially amenable and usable for preparing the corresponding pharmaceutical compositions.

The present invention provides an improved process for the preparation of substantially pure Liraglutide, wherein substantially pure material having a purity of greater than or equal to 99.5% by HPLC and meeting the quality of ICH guidelines. Liraglutide obtained by the process of the present invention is chemically stable and has good dissolution properties.

In view of the above and to overcome the prior-art problems the present inventors had now developed an improved process for the preparation of substantially pure Liraglutide, using industrially feasible and viable process, with the use of industrially friendly solvents, which does not include tedious work up and time lagging steps.

### OBJECTIVE OF THE INVENTION

The main objective of the invention relates to a process for the preparation of Liraglutide.

Yet another objective of the invention relates to an improved process for the preparation of substantially pure material having a purity of greater than or equal to 99.5% by HPLC.

Yet another objective of the invention relates to an improved process for the preparation of substantially pure material having a purity of greater than or equal to 99.5% by HPLC free of process related impurities.

### SUMMARY OF THE INVENTION

The main objective of the invention relates an improved process for preparation of highly pure liraglutide, comprising the steps of:
a) anchoring Fmoc protected Glycine with Wang Resin;
b) capping the resin obtained in step a);
c) deprotecting the amino protecting group under microwave conditions;
d) coupling the obtained Glycine-wang resin with Fmoc-Arg(Pbf)-OH in presence of coupling agent under microwave peptide synthesizer at 60-80° C. to produce Fmoc-Arg(Pbf)-Gly-wang resin;
e) deprotection of amino protecting groups followed by sequential coupling of the amino acids (14-37) as per the backbone of Liraglutide on microwave synthesizer using N-protected amino acids under microwave peptide synthesizer at 60-80° C. to produce Fmoc-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Pal-Glu(OtBu)-Glu(OtBu)-Phe-Ile-Ala-Trp(Boc)- Leu-Val-Arg(Pbf)-Gly-Arg(Pbf)-Gly-wang resin;
f) deprotection of amino protecting groups followed by sequential coupling of the amino acids (7-13) as per the backbone of Liraglutide on manual synthesizer using N-protected amino acids at 20-30° C.; followed by cleavage of the resin to obtain crude Liraglutide; and
g) purification of crude Liraglutide using preparative HPLC to obtain pure Liraglutide. Another objective of the invention relates to an improved process for preparation of highly pure Liraglutide, wherein purification had been performed using preparative HPLC using C8 or C18 column using buffer containing ion pair reagent.

### DETAILED DESCRIPTION OF THE INVENTION

The present invention relates to an improved process for solid phase synthesis of highly pure Liraglutide, comprises reacting protected glycine with resin; in the presence of an activating/coupling agent and a solvent selected from the group consisting of DMF, DCM, NMP, Acetonitrile, TFA, Piperdine, Pyridine, Diethyl Ether, Diisopropyl Ether, Methyl tertiary Butyl Ether, Ethyl acetate, Dimethyl sulphoxide, Diisopropyl ethylamine, hexane, water and combination thereof; to form Fmoc-Gly-resin and followed by capping with acetic anhydride in pyridine.

The resin used in the present invention involves the use of Wang resin, which is employed as the resin solid phase support, and said Fmoc-Gly-resin with substitution degree in the range from 0.3 to 0.5 mmol/g.

The activating/coupling agent used in the present invention selected from DIC(Diisopropyl carbodiimide), DIPEA, HOBt·H2O, TBTU, DCC (N,N′-Dicyclohexyl carbodiimide), Ethyl cyano(hydroxyimino)acetate, DIPEA(Diisopropylethylamine), HCTU(O-(1H-6-Chloro benzo triazole-1-yl)-1,1,3,3-tetramethyluronium hexafluoro phosphate), HATU (1-[Bis (dimethyl amino)methylene]-1H-1,2,3-triazolo[4,5-b]pyridinium3-oxide hexafluoro phosphate.

Capping for selected amino acids had been performed under acetic anhydride and DIPEA conditions using solvents selected from dichloromethane, dimethylformamide, isopropyl alcohol and methyl tert-butyl ether.

The obtained capped Fmoc-Gly-resin undergo deprotection in microwave peptide synthesizer in presence of 16% piperidine in DMF at 60-80° C., followed by coupling with Fmoc-Arg(Pbf)-OH in presence of coupling agent under microwave peptide synthesizer at 60-80° C. to produce Fmoc-Arg(Pbf)-Gly-wang resin.

The obtained Fmoc-Arg(Pbf)-Gly-wang resin undergo subsequent deprotection of amino protecting groups followed by sequential coupling of the amino acids (14-37) as per the backbone of Liraglutide on microwave synthesizer using N-protected amino acids under microwave peptide synthesizer at 60-80° C.; wherein purified Fmoc-Lys(Pal-Glu(OtBu)-OH is employed in place of Lysine; in presence of activating agent selected from DIC(Diisopropylcarbodiimide), DCC (N,N′-Dicyclohexylcarbodiimide) Ethylcyano (hydroxyimino) acetate, DIPEA (Diisopropyl ethylamine), HCTU(O-(1H-6-Chlorobenzo triazole-1-yl)-1,1,3,3-tetramethyl uronium hexafluoro phosphate), HATU (1-[Bis(dimethylamino)methylene]-1H-1,2,3-triazolo[4,5-b]pyridinium3-oxide hexafluoro phosphate; and solvent selected from DMF, DCM, NMP, Acetonitrile, TFA, Piperdine, Pyridine, Diethyl Ether, Diisopropyl Ether, Methyl tertiary Butyl Ether, Ethyl acetate, Dimethyl sulphoxide, Diisopropyl ethylamine, hexane, water and combination thereof; to produce Fmoc-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Pal-Glu(OtBu)-Glu(OtBu)-Phe-Ile-Ala-Trp(Boc)-Leu-Val-Arg(Pbf)-Gly-Arg(Pbf)-Gly-wang resin.

The obtained Fmoc-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Pal-Glu(OtBu)-Glu(OtBu)-Phe-Ile-Ala-Trp(Boc)-Leu-Val-Arg(Pbf)-Gly-Arg(Pbf)-Gly-wang resin undergo deprotection in microwave peptide synthesizer in presence of 16% piperidine in DMF at 60-80° C., followed by sequential coupling of the amino acids (7-13) as per the backbone of Liraglutide in manual/regular peptide synthesizer at 20-30° C. using N-protected amino acids Fmoc-Thr(tBu)-OH, Fmoc-Phe-OH, Fmoc-Thr(tBu)-OH, Fmoc-Gly-OH, Fmoc-Glu(OtBu)-OH, Fmoc-Ala-OH, Boc-His(Trt)-OH, wherein in the coupling and deprotection reactions had been performed in manual reaction conditions, whereas capping was performed for Boc-His(Trt)-OH at 7^{th} positions and Fmoc-Thr(Otbu)-OH 11^{th} positions at 20-30° C.; in presence of coupling agent selected from DIC(Diisopropyl carbodiimide), DIPEA, HOBt·H2O, TBTU, DCC (N,N′-Dicyclohexyl carbodiimide), Ethyl cyano(hydroxyimino)acetate, DIPEA(Diisopropylethylamine), HCTU(O-(1H-6-Chloro benzo triazole-1-yl)-1,1,3,3-tetramethyluronium hexafluoro phosphate), HATU (1-[Bis (dimethyl amino)methylene]-1H-1,2,3-triazolo[4,5-b]pyridinium3-oxide hexafluoro phosphate; the solvents are selected from DCM, DMF, NMP, acetonitrile, TFA, piperidine, pyridine, diethylether, diisopropylether, methyl tertiary butyl ether, ethylacetate, dimethyl sulphoxide, diisopropyl ethylamine hexane, water, isopropyl alcohol, methanol, N-propanol and combination thereof; followed by cleavage of the resin to obtain crude Liraglutide. The removal of protective groups and cleavage of resin, the cleaving agents are selected from TIPS, TFA, Phenol, water and precipitation using MTBE.

Cleavage and deprotection is one of the most crucial potential problems steps in peptide synthesis. The treatment of a peptidyl resin with a cleavage cocktail is not one simple reaction, but a series of competing reactions. Unless suitable reagents and reaction conditions are selected, the peptide can be irreversibly modified or damaged. The goal of cleavage/deprotection is to separate the peptide from the support while removing the protecting groups from the side-chains. This should be done as quickly as possible to minimize the exposure of the peptide to the cleavage reagent. The peptide is then recovered from the reaction mixture and analyzed.

The obtained Liraglutide undergo final purification and lyophilization; wherein the purification is performed by a reverse-phase high performance liquid chromatography using a reverse-phase C8 or C18 column using buffer containing ion pair reagent, wherein in Buffer is selected from MAP (Monobasic Ammonium Phosphate), HSA (Heptane sulfonic acid sodium salt anhydrous) in Water at pH 2.5 with ortho phosphoric acid and buffer B is Acetonitrile:Methanol:Propanol.

In addition, buffer used in the purification may also selected from ammonium salts selected from selected from ammonium acetate, ammonium chloride, ammonia, ammonium bicarbonate, ammonium carbonate or combination thereof; and ammonium buffers in first purification and followed by 0.1% TFA in water, acetonitrile or mixture thereof.

The prior art processes involve the fragment condensation into each of solid phase segment needed excess fragments to condensation, which results in the serious waste of peptide fragments, resulting in the high cost of synthesis. The same solid phase segment condensation needed synthesis in multiple reactions and it condensation having limitation in resin substitution, and it generate large amount of waste. Also, the solid phase fragment condensation method of synthesis generates impurities of fragments during the condensation due to unreacted fragments. To overcome these problems the present inventors developed a process for the synthesis of Liraglutide, which is industrially feasible and free of process related impurities.

The prior art processes involve the sequential coupling of solid-phase peptide synthesis of liraglutide, using 2-chlorotrityl resin as a solid-phase carrier, using DIC/HOBt as a condensing agent on specific microwave reaction technology. The use of CTC resin, which is very sensitive. The temperature and humidity during the reaction, as well as the swelling and shrinking of the resin between washes, can affect the stability of the bond between the peptide chain and the CTC resin, which can give variation of the yield of the crude peptide. The process is not yet all feasible at commercial scale and involves more purifications.

The prior art processes involve the lysine, which is protected by the ivDde/Dde side chain, after completion of linkage of liraglutide peptide sequence, the ivDde/Dde group had been deprotected, followed by inclusion of Palmitoyl side chain.

Also, the solid phase fragment condensation method of synthesis generates impurities of fragments during the condensation due to unreacted fragments. To overcome these problems the present inventors developed a process for the synthesis of Liraglutide, which is industrially feasible and free of process related impurities. The comparative study of the prior art process with the present process in view of impurity profile had been summarized as below: The listed purities are after 1” stage of purification:

| | Liraglutide Purity | | | | |
|---|---|---|---|---|---|
| | (After1^{st} stage of | | | | |
| Batch technology | purification) | Front Impurity | | Back impurity | |
| Hybrid technology | 95.95% | 0.05% | 0.21% | 0.44% | 0.39% |
| (Microwave + Manual) | | | | | |
| Microwave technology | 95.08% | 0.26% | 079% | 1.09% | 0.35% |
| (As per prior art) | | | | | |

The process related impurities that appear in the impurity profile of the Liraglutide may be substantially removed by the process of the present invention resulting in the formation of substantially pure Liraglutide, which meets the ICH guidelines.

The merit of the process according to the present invention resides in that product isolated after drying is stable and having a purity of greater than or equal to 99.5% purity by HPLC, which was not disclosed in any of the prior-art. The product obtained as per the present invention is highly pure than the any of the prior-art products obtained. Still now no-publication disclosed a purity of 99.5% with purification yield of around 12%.

Solubility is one of the important parameters to achieve desired concentration of drug in systemic circulation for achieving required pharmacological response. Poorly soluble drugs often require high doses to reach therapeutic plasma concentrations after subcutaneous administration. Low solubility is the major problem encountered with formulation development of new chemical entities as well as generic formulation development. Most of the drugs are either weakly acidic or weakly basic having poor solubility. The improvement of drug solubility thereby its oral bio-availability remains one of the most challenging aspects of drug development process. The enhancement in the purity of liraglutide, which is free of process related impurities inherently, increases the solubility of liraglutide, which plays a major role for enhancement of drug activity.

The present invention also relates to a process for the preparation of liraglutide, which is substantially pure having a purity of greater or equal to 99.5% and meeting the ICH guidelines. Further, the liraglutide obtained as per the present process is found devoid of any other process related impurities and is adequately stable to handle and store for longer time (at least up to more than 6 months) without any significant or measurable change in its morphology and physicochemical characteristics.

The following examples illustrate the nature of the invention and are provided for illustrative purposes only and should not be construed to limit the scope of the invention.

### EXAMPLES

### Example-1

### Preparation of Fmoc-Lys-(Glu (N^{a}-Palmitoyl)-OtBu)-OH

### Step 1: Synthesis of Palmitoyl-OSu Activated Ester

Weigh 256.42 g of n-hexadecanoic acid (1.0 mol), add 115.40 g of HOSu (N-Hydroxy succinimide) (1.0 mol) to 1500 ml of Ethyl acetate in a round bottom flask, and add 206 g of DCC (1.0 mol) at room temperature, stir the mixture for 12 hours. The reaction solution was filtered; the mother liquid was distilled and found oily solid, dissolved the oily solid in n-hexane, filtered and dried to yield Palmitoyl-OSu activated ester.

### Step 2: Synthesis of Palmitoyl-Glu-OtBu

101.62 g of H-Glu-OtBu (0.5 mol) and Pamitoyl Osu 176.75 gm (0.5 mol) was dissolved in a 500 ml of DMF, to the solution add 129.25 g of diisopropylethyl amine, and the reaction was allowed for stirring 12 hours to 15 hours at room temperature. After completion of the reaction, add 7 volumes of water and the pH was adjusted to 3-3.5 using 10% dilute hydrochloric acid. A white precipitate was obtained and filtered. The resulting white precipitate was recrystallized using 60 ml ethyl acetate and the solid Palmitoyl-Glu-OtBu is dried.

### Step 3: Synthesis of Palmitoyl-Glu (OSu)-OtBu

88.33 g of Palmitoyl-Glu-OtBu (0.2 mol), 27.62 g of HOSu (N-Hydroxy succinimide) (0.2 mol) was added to a reaction flask containing 500 ml of Ethyl acetate, 49.51 g of DCC (0.2 mol). The obtained reaction mixture was stirred for 12 hours, at room temperature. The reaction solution filtered, mother liquor dried into rotary evaporator, recrystallized in n-hexane 3 times, to get Palmitoyl-Glu (OSu)-OtBu activated ester.

### Step 4: Synthesis of Fmoc-Lys-(Glu (N^{a}-Palmitoyl)-OtBu)-OH

36.74 g of Fmoc-Lys-OH (0.1 mol) and 53.87 g of Palmitoyl-Glu (OSu)-OtBu was weighed. was added in to a reaction flask containing 100 ml of DMF. To the solution, 2.5 mol of diisoropylethylamine was added, and the reaction mixture was stirred for 12 hours to 15 hours at room temperature. To the obtained reaction mixture, add 7 volumes of water and then the pH was adjusted to 3-3.5 using HCl solution. A precipitate was obtained and filtered. The resulting precipitate was recrystallized in n-hexane. The solid was dried to yield Fmoc-Lys-(Glu (N^{a}-Palmitoyl)-OtBu)-OH.

### Yield: 60%

### Example-2: Anchoring Fmoc Protected Glycine with Wang Resin

In a round bottom flask 5 g of wang resin suspend in MDC 50 ml and kept for swelling for 4 to 5 hours, in another flask dissolve Fmoc-Gly-OH 1.5 to 2.5 equivalents (relative to resin) in 20 ml DMF and addition of same equivalent of Oxymapure and kept for stirring, In another separate flasks dissolve 0.1 equivalent DMAP in 20 ml DMF. Add 1 equivalent of DIC in the amino acid mixture prior to addition in resin flaks. Finally, DMAP solution addition and keep the resin flaks under stirring for 3 to 5 hours at room temperature to yield Fmoc-Gly-OH Wang resin.

### Example-3: Capping of Unreacted Resin

Charge 100 gm of Fmoc-Gly-Wang resin which have substitution about 0.3 to 0.5 mmole/gm in to a SPPS reactor. The resin swelling is completed using 10 ml/gm of dichloromethane followed by dimethylformamide. To the obtained reaction mass 51.05 g of acetic anhydride (5 molar equivalents) had been added, followed by addition 64.50 g of Diisopropylethylamine (5 molar equivalents) and dichloromethane (6 ml/gm) at 25-35° C., stir the reaction mass for 60 minutes to 90 minutes, followed by washing with Dimethylformamide (5 ml/gm of resin 2 times), Isopropyl alcohol (5 ml/gm of resin; 2 times) and Methyl tertiary butyl ether (5 ml/gm of resin; 3 times). Filter the peptidyl resin from SSPS reactor and dry for 12 hours to 14 hours at 25-35° C.

### Example-4

### Deprotection:

Charge dimethyl formamide (10 ml/gm) in to microwave peptide synthesizer containing Fmoc-Gly-Wang resin (77 gm) and swelled for 30 min to 40 minutes. To the obtained reaction mass 16% piperidine (6 ml/gm) in dimethylformamide was added and the microwave peptide synthesizer was maintained at a temperature 65 to 70° C. for 4 min to 5 min. The obtained reaction mass was washed with dimethylformamide (7 ml/gm; 4 times) and the obtained Gly-Wang resin is used for next step of coupling.

### Example-5

### Preparation of Liraglutide

### Coupling (14-37):

Dimethyl formamide (465 ml) was added in to the obtained Gly-Wang resin (Quantity) in a microwave peptide synthesizer. To the obtained reaction mass Fmoc-Arg(Pbf)-OH (48.66 gm), HObt·H2O (11.48 gm), DIC (17.55 ml) were added and the contents stirred for 10 min to 15 minutes at temperature 70 to 75° C. After completion of the reaction the solution was drained and washed with dimethyl formamide (7 ml/gm). The efficacy of the coupling and deprotection is monitored by the Kaiser Ninhydrin test, the coupling of the reaction is repeated if Kaiser test positive.

### Subsequent Deprotection and Coupling Under Microwave Conditions:

Material obtained from step II is proceeded for sequential coupling on microwave of the amino acids as per the backbone of Liraglutide that is Fmoc-Gly-OH (Gly), Fmoc-Arg(Pbf)-OH (Arg),Fmoc-Val-OH (Val), Fmoc-Leu-OH (Leu), Fmoc-Trp(Boc)-OH (Trp), Fmoc-Ala-OH (Ala), Fmoc-Ile-OH (Ile), Fmoc-Phe-OH (Phe), Fmoc-Glu(OtBu)-OH (Glu), Fmoc-Lys(Pal-Glu(OtBu))-OH, Fmoc-Ala-OH (Ala), Fmoc-Ala-OH (Ala), Fmoc-Gln(Trt)-OH (Gln), Fmoc-Gly-OH, Fmoc-Glu (OtBu)-OH, Fmoc-Leu-OH, Fmoc-Tyr (tBu)-OH, Fmoc-Ser(tBu)-OH, Fmoc-Ser(tBu)-OH (Ser), Fmoc-Val-OH, Fmoc-Asp (OtBu)-OH (Asp), Fmoc-Ser (tBu)-OH.

### Deprotection:

To the peptidyl chain obtained above, Fmoc deprotection had been performed following the process as disclosed in example 3, followed by washing with dimethyl formamide (quantity, 2 times), isopropyl alcohol (quantity, 2 times), methyl tertiary butyl ether (quantity, 3 times), which is removed from microwave synthesizer and dried for 8 hrs to 10 hours at 30 to 35° C.

### Yield: 91.0%

### Coupling (7 to 13):

To the obtained peptidyl material obtained from step II is proceeded for sequential coupling on regular SPPS. Charge Fmoc-Thr(Otbu)-OH (29.8 gm), TBTU (24.07 g) were charged in to a reaction flask containing DMF (9 ml/gm). Charge Diisopropyl ethylamine in to the obtained reaction mass. The obtained solution was slowly added to the solution containing peptidyl resin. The obtained reaction mass was stirred for 2 hours to 2 hours 30 minutes at 25±5° C. and the completion of the reaction was monitored by Kaiser test.

### Deprotection (7 to 13):

Charge dimethyl formamide (9 ml/gm) in to reaction flask containing Fmoc-Thr(Otbu)-xxx-xxx-Wang resin ( . . . gm) and swelled for 30 min to 40 minutes. To the obtained reaction mass 20% piperidine (9 ml/gm) in dimethylformamide was added and the reaction was stirred for 15 minutes to 20 minutes at a temperature ambient temperature. The obtained reaction mass was washed with dimethylformamide (2 times) and isopropyl alcohol (2 times) the obtained Gly-Wang resin is used for next step of coupling.

### Subsequent Deprotection and Coupling Under Regular (Manual) Conditions (7-12):

Material obtained from step II is proceeded for sequential coupling on regular (Manual) conditions of the amino acids as per the backbone of Liraglutide that is Fmoc-Thr (tBu)-OH, Fmoc-Phe-OH, Fmoc-Thr (tBu)-OH, Fmoc-Gly-OH, Fmoc-Glu (Otbu)-OH, Fmoc-Ala-OH and Boc-His(trt)-OH.

During the course of sequential coupling of amino acids as per the backbone of Liraglutide some of the amino acid not couples completely and hence additional reaction parameters are need, at the stage of coupling of amino acids particularly position at 7, 11 and amino acids coupling followed by capping was performed to minimize the impurities.

Capping procedure: 5 equivalent of Acetic anhydride,5 Equivalent of DIPEA in DCM as 9 ml/gm of resin and stir for 30 min and drained off.

Wash resin with 2 times with DMF, 2 Times with IPA and 2 Times with DMF.

Isolation of resin: After completion of sequence resin was washed with 2 times with DMF,2 Times with IPA and 3 Times with MTBE.

Resin was kept for drying under vacuum for 16 hrs. at temperature of 30±5° C.

### Yield of Peptidyl Resin: 90%

### Step VI Cleavage of Peptide from Resin

The peptide-resin obtained from the synthesis processed for the cleavage of peptide from resin as: 10 gram of peptide-resin taken in round bottom flaks and added 100 ml cocktail mixture consisting of TFA/TIPS/Water/Phenol (85.0%/5%/5%/5%) and stirred for 2 hours and 30 min at room temperature. The reaction mixture was filtered by sintered disk and resin washed with 1 ml/gm of peptidyl resin with TFA. The obtained filtrate was added into chilled 50 times of peptidyl resin methyl tertiary butyl ether under stirring for 60 minutes. The precipitate obtained is filtered, washed with ethyl tertiary butyl ether and dried at 25 to 30° C. for 16 hrs to obtain a dry crude Liraglutide.

Crude purity about 55 to 60%.

### Yield: 80%

### Example 6

### Purification of Crude Liraglutide

### Step I: Purification by Prep HPLC

Crude Liraglutide was dissolved in dilute ammonium bicarbonate solution containing 10-30% acetonitrile at a concentration of 10−100 mg/ml and was loaded onto 100-10-C18 column. This was followed by 3 CV (Buffer A wash) of the diluent. The bound Liraglutide was eluted using a step gradient of the mobile phase (A: 0.1% TFA in water contain 30-40% of Organic, B: 0.1% TFA in ACN:IPA). Fractions having purity >90% was pooled for next stage purification.

### Step III: Purification I by Prep HPLC

For Next stage purification dilute main pool obtained from step 1 to equal amount of purified water and load on 100-1.0-C18 column. The bound Liraglutide was eluted using a step gradient of the mobile phase (A: Phosphate buffer containing ion pair reagent in water, B: ACN: N-propanol: Methanol 60:20:20). Fractions having purity >99.0% and single impurity NMT 0.4% was pooled for next stage purification.

### Step III: Purification I by Prep HPLC

Obtained fraction from step II was diluted with equal amount of purified water and loaded on was loaded onto 100-10-C18 column purified Liraglutide eluted with 0.1M of ammonium bicarbonate containing 20% Acetonitrile and buffer B: ACN contain buffer A (80:20%). Product was eluted by applying a gradient. Detection wavelength was kept at 210 nm. The chromatographic temperature was kept ambient. Individual fractions were collected and analyzed for purity. Fractions with purity of >99.0% were pooled and distilled at 35° C. to remove organic solvent was subjected to Lyophilization. HPLC purity of the lyophilized powder was >99% with no know impurity more than 0.30%.

### Step IV: Lyophilization to Get Pure Solid Liraglutide

The aqueous solution obtained from step III is freeze gradually −20° C. degree and then −40° C. prior to Lyophilization. Lyophilization is carried out at −50 to −55° C. for 24 to 96 hours under vacuum. 8.0-gram pure solid Liraglutide obtained after the Lyophilization.

### HPLC Purity: 99.7%

### Yield: 12%

We claim:

1) An improved process for preparation of highly pure liraglutide, comprising the steps of:
a) anchoring Fmoc protected Glycine with Wang Resin;
b) capping the resin obtained in step a);
c) deprotecting the amino protecting group under microwave conditions;
d) coupling the obtained Glycine-wang resin with Fmoc-Arg(Pbf)-OH in presence of coupling agent under microwave peptide synthesizer at 60-80° C. to produce Fmoc-Arg(Pbf)-Gly-wang resin;
e) deprotection of amino protecting groups followed by sequential coupling of the amino acids (14-37) as per the backbone of Liraglutide on microwave synthesizer using N-protected amino acids under microwave peptide synthesizer at 60-80° C. to produce Fmoc-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Pal-Glu(OtBu)-Glu(OtBu)-Phe-Ile-Ala-Trp(Boc)- Leu-Val-Arg(Pbf)-Gly-Arg(Pbf)-Gly-wang resin;
f) deprotection of amino protecting groups followed by sequential coupling of the amino acids (7-13) as per the backbone of Liraglutide on manual synthesizer using N-protected amino acids at 20-30° C.; followed by cleavage of the resin to obtain crude Liraglutide; and
g) purification of crude Liraglutide using preparative HPLC to obtain pure Liraglutide.

2) An improved process for preparation of highly pure liraglutide as claimed in claim 1, wherein capping for selected amino acids had been performed under acetic anhydride and DIPEA conditions using solvents selected from dichloromethane, dimethylformamide, isopropyl alcohol and methyl tert-butyl ether.

3) An improved process for preparation of highly pure liraglutide as claimed in claim 1, wherein deprotection had been performed using 16% Piperidine in DMF.

4) An improved process for preparation of highly pure liraglutide as claimed in claim 1, wherein coupling reactions have been performed using coupling agents selected from DIC(Diisopropyl carbodiimide), DIPEA, HOBt·H2O, TBTU, DCC (N,N′-Dicyclohexyl carbodiimide), Ethyl cyano(hydroxyimino)acetate, DIPEA(Diisopropylethylamine), HCTU(O-(1H-6-Chloro benzo triazole-1-yl)-1,1,3,3-tetramethyluronium hexafluoro phosphate), HATU (1-[Bis (dimethyl amino)methylene]-1H-1,2,3-triazolo[4,5-b]pyridinium3-oxide hexafluoro phosphate.

5) An improved process for preparation of highly pure liraglutide as claimed in claim 1, wherein the amino acids (14-37) as per the backbone of Liraglutide selected from Fmoc-Gly-OH, Fmoc-Arg(Pbf)-OH, Fmoc-Val-OH, Fmoc-Leu-OH, Fmoc-Trp(Boc)-OH, Fmoc-Ala-OH, Fmoc-Ile-OH, Fmoc-Phe-OH, Fmoc-Glu(OtBu)-OH, FmocLys(Pal-Glu(OtBu))-OH, Fmoc-Ala-OH, Fmoc-Ala-OH, Fmoc-Gln(Trt)-OH, Fmoc-Gly-OH, Fmoc-Glu(OtBu)-OH, Fmoc-Leu-OH, FmocTyr(tBu)-OH, Fmoc-Ser(tBu)-OH, Fmoc-Ser(tBu)-OH, Fmoc-Val-OH, Fmoc-Asp(OtBu)-OH, Fmoc-Ser(tBu)-OH, wherein in the coupling and deprotection reactions had been performed in Microwave reaction conditions.

6) An improved process for preparation of highly pure Liraglutide as claimed in claim 1, wherein the amino acids (7-13) as per the backbone of Liraglutide selected from Fmoc-Thr(tBu)-OH, Fmoc-Phe-OH, Fmoc-Thr(tBu)-OH, Fmoc-Gly-OH, Fmoc-Glu(OtBu)-OH, Fmoc-Ala-OH, Boc-His(Trt)-OH, wherein in the coupling and deprotection reactions had been performed in manual reaction conditions, whereas capping was performed for Boc-His(Trt)-OH at 7^{th} positions and Fmoc-Thr(Otbu)-OH 11^{th} positions.

7) An improved process for preparation of highly pure Liraglutide as claimed in claim 1, the solvents are selected from DCM, DMF, NMP, acetonitrile, TFA, piperidine, pyridine, diethylether, diisopropylether, methyl tertiary butyl ether, ethylacetate, dimethyl sulphoxide, diisopropyl ethylamine hexane, water, isopropyl alcohol, methanol, N-propanol and combination thereof.

8) An improved process for preparation of highly pure Liraglutide as claimed in claim 1, the removal of protective groups and cleavage of resin, the cleaving agents are selected from TIPS, TFA, Phenol, water and precipitation using MTBE.

9) An improved process for preparation of highly pure Liraglutide, wherein purification had been performed using preparative HPLC using C8 or C18 column using buffer containing ion pair reagent.

10) An improved process for preparation of highly pure Liraglutide as claimed in claim 9, wherein in Buffer is selected from MAP (Monobasic Ammonium Phosphate), HSA (Heptane sulfonic acid sodium salt anhydrous) in Water at pH 2.5 with ortho phosphoric acid and buffer B is Acetonitrile:Methanol:Propanol.
